(19) United States
(12) Patent Application Publication
(10) Pub. No.: US 2026/0060953 A1
(43) Pub. Date: Mar. 5, 2026

(54) ANESTHETIC HEALING SPRAY CAPABLE OF BEING SPRAYED UPSIDE DOWN

(71) Applicant: Fridababy, LLC, Miami, FL (US)

(72) Inventors: Janette Marie VILLALOBOS, Miami Springs, FL (US); Jenna Christine HUNTER, New Port Richey, FL (US); Megan Ann RUMLEY, Hillsborough, NJ (US); Chelsea HIRSCHHORN, Miami Beach, FL (US)

(21) Appl. No.: 19/314,616

(22) Filed: Aug. 29, 2025

Related U.S. Application Data
(60) Provisional application No. 63/689,842, filed on Sep. 2, 2024.

(51) Int. Cl. A61K 31/245; A61K 9/00; A61K 31/01; A61K 31/047; A61K 31/404; A61K 47/34; A61M 11/00

(52) U.S. Cl. CPC A61K 31/245; A61K 9/0014; A61K 31/01; A61K 31/047; A61K 31/404; A61K 47/34; A61M 11/00

(57) ABSTRACT

A sprayable composition that includes up to 10% benzocaine, a skin protectant selected from the group consisting of petrolatum, mineral oil, glycerin, and a propellant. The benzocaine provides a cooling and/or numbing sensation to a treated area, while the skin protectant enhances healing of skin. The composition may be sprayed unto the perineum area, which can become sore, bruised, or even torn during the baby delivery process. The composition may also be sprayed from a container when the container is upside-down.

### CROSS-REFERENCE TO RELATED APPLICATION

The present application claims the benefit of U.S. Provisional Patent Application No. 63/689,842, filed on Sep. 2, 2024, and titled “ANESTHETIC HEALING SPRAY CAPABLE OF BEING SPRAYED UPSIDE DOWN,” the disclosure of which is expressly incorporated by reference in its entirety.

### FIELD

Aspects of the present disclosure generally relate to an anesthetic healing spray, and more specifically to an anesthetic healing spray that is capable of being sprayed upside down from a container.

### BACKGROUND

Postpartum perineal pain is a widespread and often challenging condition that many women face following childbirth, particularly after a vaginal delivery. The perineum, which is the area between the vaginal opening and the anus, can endure significant stress during the birthing process, leading to various degrees of pain and discomfort.

During childbirth, especially when delivering vaginally, the perineum is subjected to intense pressure and stretching as the baby passes through the birth canal. This pressure can result in the perineum becoming sore, bruised, or even torn. These tears can range in severity, from minor surface tears that only affect the skin to more severe lacerations that extend into the deeper muscle layers of the perineum. In some cases, these natural tears may require stitches to heal properly, contributing to the overall discomfort.

An episiotomy, which is a surgical incision made by a healthcare provider to enlarge the vaginal opening during delivery, can also contribute to postpartum perineal pain. Although episiotomies are less common today due to increased awareness of their potential complications, they may still be performed in certain situations to facilitate delivery. The incision, along with the healing process, can cause significant discomfort in the postpartum period.

In addition to tears and episiotomies, the physical strain and pressure exerted during childbirth can lead to swelling and bruising in the perineal area. This swelling is often a natural response to the trauma experienced during delivery, but it can cause prolonged discomfort and make it difficult for new mothers to sit, walk, or even perform basic self-care tasks.

Another common issue that can exacerbate perineal pain after childbirth is the development of hemorrhoids. Hemorrhoids are swollen veins in the rectal area, often caused by the increased pressure and straining during labor. These can be particularly painful and add to the overall discomfort in the perineal region.

The intensity of postpartum perineal pain can vary widely from one woman to another, depending on several factors, such as the extent of any tears, whether an episiotomy was performed, the presence of swelling or bruising, and the development of hemorrhoids. For some, the pain may be mild and manageable with over-the-counter pain relief and home care, while others may experience more severe pain that requires medical intervention.

### SUMMARY

Various aspects of the present disclosure relate broadly to sprayable compositions for providing pain relief and skin protectant to promote healing. Following delivery, the perineal area may be sore, bruised, or torn, and the sprayable composition provides for a hands-free, hygienic application method. For optimal comfort, these sprays should be designed to function upside-down, ensuring continuous delivery of the formulation. By utilizing these ingredients and application methods, postpartum perineal pain can be effectively managed, promoting faster healing and providing much-needed pain relief.

Nozzle and spray failures are very common in aerosols. Formula instability can cause an aerosol nozzle to fail or sputter due to several related mechanisms. Instability in the formula can lead to precipitation, thickening or thinning inside the nozzle or fluid pathways. This restricts or intermittently blocks fluid flow, causing sputtering or inconsistencies in spray patterns. Instability that increases viscosity or creates solids can cause inadequate fluid pressure or flow through the nozzle. Inadequate fluid pressure or flow allows propellant to mix irregularly with the fluid inside the nozzle, leading to sputtering or spitting that disrupts smooth spraying. Components in an unstable formulation may settle or dry on the nozzle tip, changing the surface and interfering with proper atomization. This causes erratic spray flow and sputtering. Changes in fluid properties like surface tension and viscosity caused by instability impact the atomization mechanism, leading to fluctuating droplet formation and thus sputtering outputs.

Surface tension and viscosity affect nozzle performance and can cause failure or sputtering by disrupting the atomization process. If surface tension is too high, the liquid resists breaking up into fine droplets, causing larger droplets or irregular spray patterns, which can lead to sputtering or spitting from the nozzle. Conversely, if the surface tension is too low, it can cause unstable spray formation. In aerosols, a balanced surface tension may maintain smooth spray breakup and droplet formation.

Increased viscosity makes it harder for the formulation to flow easily through the narrow nozzle orifices, reducing flow rate and increasing the pressure needed to eject the liquid. High viscosity delays the breakup of the liquid stream into fine droplets, causing longer liquid sheets or filaments and larger droplets. This can cause sputtering or inconsistent spray patterns as the nozzle struggles to properly atomize the thick fluid. Highly viscous fluids also tend to clog or cause flow irregularities in the nozzle. If the formula is too thin, the propellant overwhelms product, creating an inconsistent spray pattern. Sputtering is usually a symptom of phase separation, precipitation, viscosity drift, or propellant/product incompatibility.

Additionally, sprayable compositions intended for postpartum recovery should be able to spread on the skin to provide sufficient coverage of the perineal area. However, this can be difficult because of the variability in the surface properties of the skin. If the surface tension of the sprayable composition is too high, the sprayable composition can bead-up on the skin and not properly spread, impacting the ability of the composition to deliver its intended benefits.

Surprisingly, it was found that the inclusion of certain surface tension modifiers in the sprayable composition can improve the coverage on skin without lowering viscosity and disrupting stability, ensuring a consistent fine mist pattern. In particular, it was found that a sprayable composition comprising benzocaine, a skin protectant, a polyol, and a surface tension modifier, and mixtures thereof, can provide a fine mist with excellent coverage while maintaining viscosity, surface tension, and long-term stability.

One advantage to including such a surface tension modifier in the sprayable composition is that it can lower the surface tension while maintaining the viscosity and stability of the product. Another advantage is that it can provide skin conditioning benefits, along with increased skin absorption of actives, such as the benzocaine.

Without wishing to be bound by theory, it is believed that in the sprayable composition described herein, materials such as surfactants can lower the surface tension, however they can affect active solubility and stability, potentially causing benzocaine to crystalize, therefore affecting the level of benefit and causing issues on spraying such as sputtering, uneven spray, weak spray, full blockage. Solids can clog the dip tube or valve, sputtering, weak spray, or full blockage.

In an aspect of the present disclosure, the composition includes 1 to 10% benzocaine, 1 to 25% of a skin protectant selected from the group consisting of petrolatum, mineral oil, glycerin, or a combination thereof, at least 1% propellant; and an aerosol carrier comprising a polyol. Benzocaine is a lipophilic ester and has a faster onset time of about 30-60 seconds, compared to other analgesics. However, the benzocaine is provided in a relatively high concentration (compared to other local analgesics) to produce the desired pain relief effect. Given the chemical properties of benzocaine, the higher concentration that is needed, and the inclusion of a sufficient amount of skin protectant, carrier, and propellent, such a composition may not be generally suitable for dispensing in a sprayable format. The present disclosure relates broadly to such sprayable compositions that promote faster healing and provide much-needed pain relief.

The present disclosure also relates broadly to spray assemblies for use in dispensing a sprayable composition from a container. The structure of the spray assembly may be configured to be suitable for use with the particular sprayable compositions of the present disclosure. Various aspects of the present disclosure relate broadly to compositions for an anesthetic spray that may be sprayed when the spray container is upside-down. That is, when the dispensing end is below the bottom of the container. The spray formula includes an analgesic and a skin protectant along with a propellant. The present disclosure addresses the problem of providing a desired quantity of both benzocaine and skin protectant, while providing a sprayable composition, including spraying when the container holding the spray formula is in an upside down position.

### BRIEF DESCRIPTION OF THE DRAWINGS

So that the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

FIG. 1A is a perspective view of a spray assembly, in accordance with various aspects of the present disclosure.

FIG. 1B is a perspective view of the spray assembly of FIG. 1A, wherein the spray composition is being dispensed.

FIG. 2 is a diagram three images showing the operation of the spray assembly of FIGS. 1A and 1B.

FIG. 3A is a perspective view of a spray assembly, in accordance with various aspects of the present disclosure.

FIG. 3B is a cross-sectional view of the spray assembly of FIG. 3A.

FIG. 4A is a perspective view of an insert, in accordance with various aspects of the present disclosure.

FIG. 4B is a cross-sectional view of the insert of FIG. 4A.

FIG. 5 is a graph of the viscosity of a spray composition measured in relation to the revolutions per minute of the rotational viscometer.

FIG. 6 is a flow diagram illustrating an example of a process 600 for providing pain relief and skin protection, in accordance with various aspects of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

Based on the teachings, one skilled in the art should appreciate that the scope of the present disclosure is intended to cover any aspect of the present disclosure, whether implemented independently of or combined with any other aspect of the present disclosure. For example, an apparatus may be implemented, or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses, or objectives. Rather, aspects of the present disclosure are intended to be broadly applicable to different technologies, system configurations, networks, and protocols, some of which are illustrated by way of example in the figures and in the following description of the preferred aspects. The detailed description and drawings are merely illustrative of the present disclosure rather than limiting, the scope of the present disclosure being defined by the appended claims and equivalents thereof.

As used herein, the terms “components,” “ingredients,” and “materials” may be used interchangeably unless otherwise specified.

As used herein, “cosmetic agent” means any substance, as well as any component thereof, intended to be rubbed, poured, sprinkled, sprayed, introduced into, or otherwise applied to a mammalian body or any part thereof to provide a cosmetic benefit. Cosmetic agents may include substances that are Generally Recognized as Safe (GRAS) by the United States Food and Drug Administration, food additives, and materials used in non-cosmetic consumer products including over-the-counter medications. A cosmetic agent may include, but is not limited to, (i) chemicals, compounds, small or large molecules, extracts, formulations, or combi-nations thereof that are known to induce or cause at least one effect (positive or negative) on skin tissue; (ii) chemicals, compounds, small molecules, extracts, formulations, or combinations thereof that are not known have an effect on skin tissue and are discovered, using the provided methods and systems, to induce or cause an effect on skin tissue; and (iii) a compound or combination of compounds that, when applied to skin, provide an acute and/or chronic benefit to skin or a type of cell commonly found therein. Cosmetic agents may regulate and/or improve skin or its associated cells (e.g., improve skin elasticity; improve skin hydration; improve skin condition; and improve cell metabolism).

As used herein, “skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firm-ness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, soothing irritated skin feel and/or improving the brightness, radiancy, or translucency of skin; preventing damage to skin via antioxidant approaches, including UV A and UV B induced damage, preventing formation of comedones, balancing the skin microbiome or preventing acne.

As used herein, “skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

As used herein, the term “anesthetic topical application” means to apply or spray the compositions of the present disclosure onto the surface of the skin, specifically the perennial area.

The phrase “dermatologically acceptable carrier,” as used herein, means that the carrier is suitable for personal care topical application to the keratinous tissue without undue toxicity, incompatibility, instability, allergic response, and the like, has good aesthetic properties, and is compatible with any additional components of the skincare composition.

As used herein, the terms “plant extract,” “natural extract,” or “extract” with reference to a plant is any material extracted from natural resources, including plants. The entire plant or any part of the plant including the bark, berries, 60 flowers, leaves, stem, stalk, peels, resins, rhizome, roots, seeds, woods, and mixtures thereof may be used for the extraction process. Extracts may be obtained using any suitable method known in the art including: milling, grinding, maceration, infusion, percolation and decoction, Soxhlet extraction, microwave-assisted extraction, ultra-sound-assisted extraction, sonication extraction, solvent extraction, accelerated solvent extraction, and supercritical fluid extraction. Suitable extraction solvents may include water, ketones, esters, C1 to C6 alcohols, hydrocarbons, and mixtures thereof.

As used herein, the term “safe and effective amount” means an amount of a material, ingredient, compound, component, or composition sufficient to significantly induce a positive benefit, but low enough to avoid serious side effects such as undue toxicity or allergic reaction.

As used herein, the term “skin” means the outermost protective covering of mammals that is composed of cells such as keratinocytes, fibroblasts, and melanocytes. Skin includes an outer epidermal layer and an underlying dermal layer. Skin may also include hair follicles, sebaceous glands, and nails, as well as other types of cells commonly associated with skin, such as, for example, myocytes, Merkel cells, Langerhans cells, macrophages, stem cells, sebocytes, nerve cells, and adipocytes.

As used herein, the terms “sensitive skin,” “hypersensitive skin,” and “challenged skin” may include irritable skin and intolerant skin. As used herein, “irritable skin” means skin that reacts through pruritus, i.e., through itching or stinging, to various factors such as the environment, emotions, foods, the wind, rubbing, shaving, hard water with a high calcium or other element concentration, temperature variations, humidity, or wool. As used herein, “intolerant skin” means skin that reacts to various factors, such as the application of cosmetic or dermatological products or soap, through sensations of overheating, tautness, pins and needs, and/or redness.

The terms “substantially free of” or “substantially free from” may be used herein. This means that the indicated material is, at the very minimum, not deliberately added to the composition to form part of it. The indicated material may be present, if at all, at a level of less than about 0.5%, or less than about 0.01%, or less than about 0.0001%, or less than about 0.000001%, or even 0%, by weight of the composition.

As used herein, “ambient conditions” refers to a temperature of about 23 degrees Celsius (° C.) and 50% relative humidity (RH).

As used herein, “viscosity” means the viscosity at about 0.10 min⁻¹ as determined by the Brookfield Viscosity Test Method, described hereafter. In some examples, the viscosity may be greater than 0.10 min⁻¹.

A surface tension modifier refers to an agent effective to reduce the surface tension of a composition of the present disclosure. Typically, a suitable surface tension modifier is used in amounts effective to reduce the surface tension of the same composition without a surface tension modifier by at least about 10 mN/m, preferably by 15 mN/m, 20 mN/m or more.

As used herein, the articles “a” and “an” are understood to mean one or more of the materials that is claimed or described, for example, “a rheology modifier” or “an active.”

As used herein, the terms “include,” “includes,” and “including” are meant to be nonlimiting.

Unless otherwise noted, all component or composition levels are in reference to the active portion of that component or composition, and are exclusive of impurities, for example, residual solvents or by-products, which may be present in commercially available sources of such components or compositions.

In the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Active and other ingredients useful herein may be categorized or described herein by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

As discussed, the intensity of postpartum perineal pain can vary widely from one woman to another, depending on several factors, such as the extent of any tears, whether an episiotomy was performed, the presence of swelling or bruising, and the development of hemorrhoids. For some, the pain may be mild and manageable with over-the-counter pain relief and home care, while others may experience more severe pain that requires medical intervention.

Topical analgesics are effective for managing postpartum perineal pain due to their ability to provide localized relief with minimal systemic side effects. Local anesthetics such as benzocaine and lidocaine are particularly effective in numbing the perineal area, thereby offering temporary relief from pain and discomfort. These anesthetics are available in various forms, including gels, sprays, and ointments.

The formulation includes skin protective ingredients such as glycerin, petrolatum, and mineral oil, which provide additional skin protectant benefits to aid the healing process. Glycerin, also known as glycerol, is a versatile compound widely used in skincare for its hydrating and healing properties. Glycerin promotes faster wound healing by creating an optimal environment for skin repair and maintaining moisture in the affected area. Glycerin is non-irritating and hypoallergenic, making it suitable for sensitive skin, and can be used on damaged or compromised skin without causing further irritation.

Petrolatum, commonly known as petroleum jelly and mineral oil, is also widely used in skincare due to its protective and healing properties. Both are occlusive substances, forming a protective barrier on the skin's surface to seal in moisture, prevent water loss, and keep the skin hydrated. Similar to glycerin, petrolatum and mineral oil may create an optimal environment for skin healing by maintaining moisture in the affected area, thereby speeding up perineal recovery. These ingredients are hypoallergenic and safe for use on sensitive skin, making them desirable in skin healing. All are included in the FDA's skin protectant monograph (FDA Monograph M016).

Considering that the perineal area may be sore, bruised, or torn after delivery, it may be desirable to use a hands-free, hygienic application method such as a spray. Postpartum sprays should be used after cleansing while using the bathroom. For optimal comfort, these sprays should be designed to function upside-down, ensuring continuous delivery of the formulation. By utilizing these ingredients and application methods, postpartum perineal pain can be effectively managed, promoting faster healing and providing much-needed pain relief.

Some conventional postpartum pain sprays, such as Dermaplast Postpartum Spray and Lansinoh Pain Relief Spray, are intended to relieve perineal pain, itching, and discomfort. However, Dermaplast does not contain any skin protectant or healing ingredients and includes acetylated lanolin alcohol, which can potentially cause skin irritation and sensitization. Similarly, Lansinoh Pain Relief Spray, while designed for postpartum care, lacks skin protectant or healing ingredients and contains alcohol, lavender oil, and peppermint oil, all of which can cause skin irritation. Additionally, Lansinoh's pump spray delivery system tends to clog and lose functionality during use.

Conventional skin protectants or barrier sprays, such as Dr. Butler's Pregnancy and Postpartum Spray, offer moisturizing, soothing, and protective benefits but do not spray reliably and lack pain relief properties. Aquaphor Healing Ointment Body Spray, although a petrolatum-based cosmetic spray, is not designed for postpartum use and does not provide pain relief.

Conventional combination lotions, such as Preparation H Rapid Relief Hemorrhoid Symptom Treatment Cream with Lidocaine and Tucks 5% Lidocaine Cream combine an analgesic and a skin protectant in a cream form, requiring application by hand.

Various aspects of the present disclosure are directed to a composition that delivers both pain relief and skin healing benefits in an upside-down sprayable format. Specifically, such aspects are directed to an anesthetic spray that delivers a gentle mist containing benzocaine (an analgesic) and a skin protectant, in an aerosol can that may be sprayed upside-down. The composition does not include alcohols and other potential irritants, which may interfere with the healing process. Potential skin irritants may include alcohols such as acetylated lanolin alcohol, lavender oil, peppermint oil, and other potentially irritating or drying components. This spray is designed to be cooling and non-irritating, providing pain relief in the perineal and anal regions postpartum while promoting skin healing by keeping the area moisturized.

The sprayable composition includes an analgesic to provide a local cooling and anesthetic effect, a skin protectant, a carrier, a skin conditioning agent, and a propellant. The present disclosure may also be described as a formulation, which includes an analgesic, a skin protectant, a skin conditioning agent, and a carrier, but it does not include the propellant. The formulation may be prepared and then combined with the propellant when the container is filled. The sprayable composition may also be referred to as the “spray” or the “pain and skin healing composition”.

In some examples, the pain and skin healing composition comprises up to 10% (20% in the formula without the propellant) benzocaine, which is an effective local anesthetic. Other analgesics may also be used, such as lidocaine, capsaicin, salicylates, and menthol.

The pain and skin healing composition also includes up to 10% (20% in the formula without the propellant) of petrolatum, mineral oil, glycerin, or a combination thereof, which serve as skin protectants. In some such examples, the composition includes up to 25% of a skin protectant. Glycerin is preferred due to its non-greasy skin feel, although mixtures of petrolatum, mineral oil, and glycerin can also be used. Nonlimiting examples of skin protectants can include allantoin, colloidal oatmeal, and dimethicone. Examples of suitable petrolatum can include: Snow White Pet-C from Calumet Specialty Products, Indianapolis, Ind. (melting range: 51-54° C.), Snow White V30 from Sonneborn, Parsippany, N.J. (melting range: 55-64° C.), Rajell WP1008AB5 Silkolene/Raj from Raj Petro Specialties P. Ltd., Mumbai, India (melting range: 65-68° C.), Rajell WP 29 AJB from Raj Petro Specialties P. Ltd., (melting range: 63-69° C.), Pet Blend 670 PG from Calumet Specialty Products, (melting range: 64-72° C.), Merkur 873 from Sasol Performance Chemicals, Hamburg, Germany (melting range: 55-64° C.), equivalents thereof, and mixtures thereof.

The composition utilizes up to 50% propellant, preferably dimethyl ether, a propellant known for its fast-drying and safer properties. The aerosol and aerosol weight percentage may be suitable for use with a specific insert and valve that reliably sprays upside-down, ensuring consistent application, as described in more detail below.

In some examples, the carrier for the composition be a surface tension modifier. The carrier may comprise a polyol selected from the group consisting of polyhydric alcohols, polyethylene glycol, PPG-3 methyl ether 1,3-butylene glycol, propylene glycol, diglycerol, polyethylene glycol ethers of glycerin (e.g., glycereth 20), glycerol monopropoxylate, glycogen, hexylene glycol, and extracts or derivatives thereof, hydrogenated starch hydrolysates, hydrolyzed mucopolysaccharides, inositol, glycosaminoglycans, methoxy PEG-10, methyl gluceth-10, methyl gluceth-20, methyl glucose, 3-methyl-1,3-butanediol, polyethylene glycol and derivatives thereof (such as PEG-15 butanediol, PEG-4, PEG-5 pentaerythitol, PEG-6, PEG-8, PEG-9), pentaerythitol, 1,2 pentanediol, PPG-1 glyceryl ether, PPG-9,2-pyrrolidone-5-carboxylic acid and its salts such as glyceryl pea, saccharide isomerate, sericin, silk amino acids, sodium acetylhyaluronate, sodium hyaluronate, sodium poly-aspartate, sodium polyglutamate, sorbeth 20, sorbeth 6, sugar and sugar alcohols and derivatives thereof such as glucose, mannose and polyglycerol sorbitol, trehalose, triglycerol, trimethyolpropane, tris (hydroxymethyl) amino methane salts, and yeast extract, and mixtures thereof. Nonlimiting examples of polyhydric alcohols may include glycerin, diglycerin, glycerol, erythritol, arabitol, xylitol, ribitol, mannitol, sorbitol, galactitol, fucitol, maltitol, mannose, inositol, triethyleneglycol, sodium pyrrolidone carboxylic acid (PCA), zinc PCA, and derivatives and mixtures thereof. In another aspect, the spray composition may include one or more surface tension modifiers, in addition to the one or more polyols.

The composition may contain additional safe and effective amounts of skin conditioning agents. Nonlimiting examples of skin conditioning agents (also referred to as “skin soothing agents”) suitable for use herein may include Aloe Barbadensis (aloc) leaf juice, Hamamelis Virginiana (witch hazel) extract, Aveena Sativa (oat) extract, green tea extract, chamomile extract, and Centella asiatica, Zingiber Officinale (ginger) root extract, panthenoic acid derivatives (including panthenol, dexpanthenol, ethyl panthenol), bisabolol, dipotassium glycyrrhizinate, pentylene glycol, 4-t-butylcyclohexanol, PEG-40 hydrogenated castor oil, hydroxyphenyl propamidobenzoic acid, trideceth-9, propylene glycol, and mixtures thereof. Nonlimiting examples of suitable commercially available preservatives include SymSitive® (pentylene glycol, 4-t-butylcyfclohexanol); SymCare® (pentylene glycol (and) 4-t-butylcyclohexanol (and) PEG-40 hydrogenated castor oil (and) trideceth-9 (and) hydroxyphenyl propamidobenzoic acid (and) propylene glycol); and SymRelief® (bisabolol (and) Zingiber ojficinale (ginger) root extract), all made available by Symrise, Holzminden, Germany). The skin conditioning agent may be provided in an amount of about 0.01 to 10%.

In known aerosol atomizers or spray cans, a liquid contained therein is held under pressure by a propellant present in the container and is expelled through a nozzle to form an atomized jet. Constituents of the spray can are the liquid and/or gaseous propellant since this generates the necessary pressure for spraying, and the actual product—the active ingredient, the preparation, which is to be sprayed. The product to be applied is gaseous or liquid and/or mixed in the can with the propellant, the propellant gas.

As a result of the internal pressure in the spray can, its contents are released precisely as aerosol if the spray head is pressed. The success of this function lies in the mixture of composition and liquid propellant inside the spray can. Some or all of the propellant is dissolved in the composition, or some of the propellant is in gaseous form as a “pressure cushion” above the active ingredient/propellant mixture. If the valve in a spray assembly is actuated, the gaseous propellant forces the contents through the valve to the outside. At this moment, the propellant evaporates in fractions of seconds and the remaining active ingredient distributes itself finely and evenly.

Some compositions cannot be mixed directly inside the can with a propellant. For example, the active ingredients/products/preparations to be discharged are often unstable towards the propellant. Some compositions, such as emulsions, high viscosity gels, or oils, can be sprayed with difficulty or not at all.

In some examples, the propellant for the composition can be dimethyl ether, n-butane, isobutane, propane, or mixtures thereof. Dimethyl ether is preferred as the propellant due to its global acceptance and fast-drying nature. In some examples, dimethyl ether is used in a 50/50 dilution with the formula to deliver a drier spray. The sprayable composition may also include preservatives, such as Pentylene Glycol and/or -t-Butylcyclohexanol. Other components may also be added to modify the solubility or dispersibility of the composition.

The insert, actuator, and valve combination are configured to reliably spray the composition with a consistent pattern and output. The formulation of the spray may be suitable for particular valves and/or inserts.

Benzocaine, being an ester and lipophilic, has a faster onset time of 30-60 seconds compared to lidocaine, making it a preferred active ingredient. However, benzocaine is required at higher concentration of 5-20% for pain relief, thus making it difficult to find a suitable carrier to solubilize both the high level of benzocaine and the skin protectant.

### Method of Making Exemplary Compositions

The compositions of the present invention may be generally prepared by conventional methods such as are known in the art of making sprayable non-aqueous compositions. Such methods typically involve mixing of the ingredients in one or more steps to a relatively uniform state, with or without heating, cooling, application of vacuum, and the like. Typically, systems that are less than 5% water are considered non-aqueous and are single phase. They may be prepared by first mixing the carriers, gently heating then adding actives such as liquids or powders, finally cooling and adding additional skin conditioning ingredients. The compositions may preferably be prepared such as to optimize stability (e.g., physical stability, chemical stability) and/or delivery of the active materials.

Table 1, below, provides the composition for several exemplary spray formulations.

TABLE 1

| | | | Example | Example | Example | Example |
|---|---|---|---|---|---|---|
| | | | 1 Wt % | 2 Wt % | 3 Wt % | 4 Wt % |
| | Compound | Function | Active | Active | Active | Active |
| 1 | Benzocaine, USP | Analgesic | 10 | 5 | 5 | 2.5 |
| 2 | Glycerin 99.7% | Skin Protectant | 10 | 12.5 | 12.5 | 22.5 |
| 3 | Petrolatum | Skin Protectant | 0 | 2.5 | 0 | 0.5 |
| 4 | Mineral Oil | Skin Protectant | 0 | 0 | 2.5 | 0 |
| 5 | Polyethylene | Carrier | Balance | Balance | Balance | Balance |
| | Glycol | | | | | |
| 7 | PPG-3 Methyl | Carrier | 3.0 | 5 | 5 | 1.5 |
| | Ether | | | | | |
| 8 | Hamamelis | Skin Soothing | 0.5 | 0 | 0 | 0 |
| | Virginiana (Witch | | | | | |
| | Hazel) Extract | | | | | |
| 9 | Aloe Barbadensis | Skin Soothing | 0 | 0.5 | 0 | 0.1 |
| | Leaf Juice | | | | | |
| 10 | Oat Extract | Skin Soothing | 0 | 0 | 0.05 | 0 |
| 11 | Pentylene Glycol | Preservative | 0.7 | 0 | 0 | 0.7 |
| 12 | 4-t- | Preservative | 0.3 | 0 | 0 | 0.3 |
| | Butylcyclohexanol | | | | | |
| 13 | Dimethyl ether | Propellent | 50 | 50 | 50 | 50 |

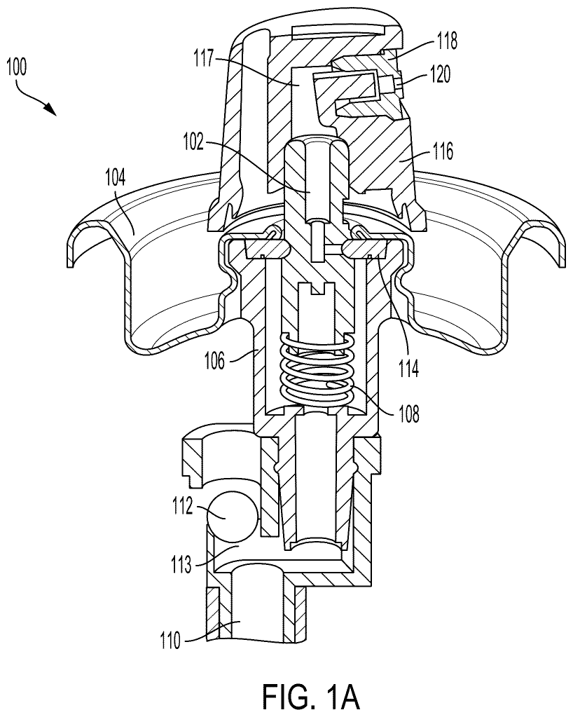

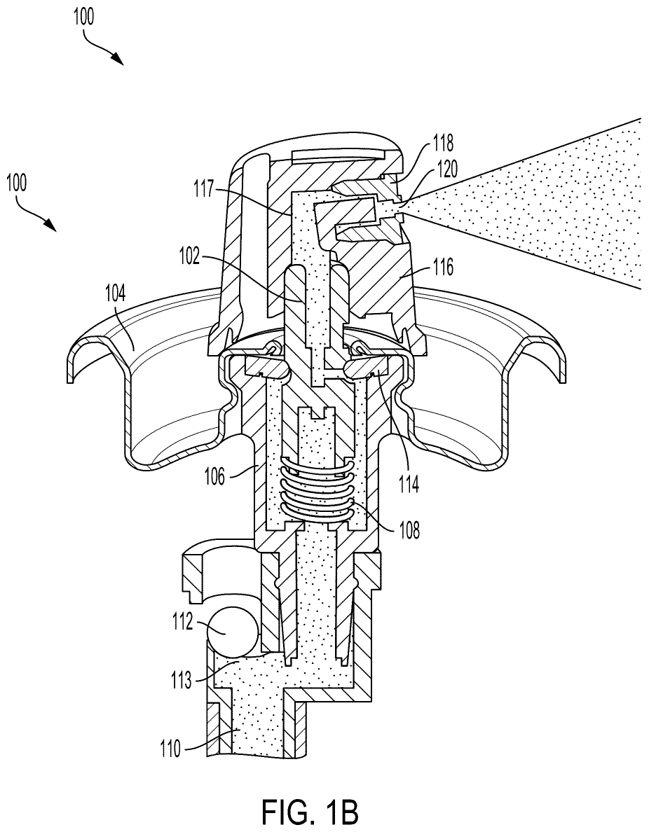

FIGS. 1A and 1B illustrate a spray assembly 100 for use in dispensing a sprayable composition. The spray assembly 100 may be specified for reliable and consistent performance. The spray assembly 100 may be coupled to a container (not shown), for example, by crimping the edges of the spray assembly to the edges of a cylindrical container. The spray assembly 100 includes a valve 102 that is disposed in a valve fixture 104. The valve 102 is also in contact with a housing 106, and a spring 108 holds the valve 102 against a gasket 114 to close the spray assembly 100 in the absence of an outside force by a user to displace the valve 102. The spray assembly 100 also includes a dip tube 110 that extends downward and into the container. The dip tube 110 may extend to a bottom of the container so substantially all the liquid content may be expelled from the container. When the spray assembly 100 is positioned upright, as shown in FIGS. 1A and 1B, ball 112 is positioned in a down position, such that the ball 112 blocks a second opening 113 in the dip tube 110. When the spray assembly 100 is rotated to be substantially upside-down, the ball 112 moves out of the second opening 113, so the spray formulation may enter the dip tube 110 via the second opening 113 and subsequently flow into the housing 106. An actuator 116 is coupled to the top of the valve 102, such that when the actuator 116 is pressed downward, the valve 102 is displaced from its resting position, and the composition flows through a gap between the valve 102 and the housing 106 and into a fluid path 117 in the actuator 116. An insert 118 is disposed is coupled to the actuator 116, and the insert 118 includes an aperture 120 through which the spray formulation passes through and is dispensed as a mist.

The valve 102 may have a height of at least 6 mm before crimping. In some configuration, the valve 102 height may be 6.10-6.96 mm before crimping. The height may vary after crimping. In some configurations, the height may be 6.93-7.95 mm after crimping. The gasket 114 may be made from materials such as, but not limited to, BUNA, neoprene, butyl, or Viton, provides a seal to prevent leakage and may have a height of at least 4 mm. In some configurations, the gasket 114 height may be 4.95-5.21 mm. The valve fixture 104 may be made from aluminum, and it may have an outer diameter of at least 32 mm and is designed to be crimped onto the container to ensure a secure fit, may have initial crimp dimensions of at least 27 mm for the valve fixture on an aluminum container. In some configurations, the outer diameter of the valve fixture 104 is 32.5 mm. Additionally, in some configurations, the initial crimp dimension may be 27.05 to 27.31 mm. In some configurations, the outer diameter may be 1.280-1.290 inches (32.51-32.77 mm). The spring 108 may be a stainless steel spring that provides the necessary force to return the valve 102 to its closed position after dispensing. Housing 106 may be made from nylon or other suitable materials. The ball 112 may be a stainless steel ball that helps control the flow of the product. The dip tube 110 may be made from a polyethylene or other suitable materials, and the dip tube 100 extends into the spray formulation that is inside the container, allowing the spray formulation to be drawn up and dispensed through the valve 102. The dip tube measurement may be specified to be at least 2 mm.

The actuator 116 may be made from a polymer, such as polypropylene (PP) or polyethylene terephthalate (PET), or another material. The actuator 116 may also include a finger pad and side grips with a textured or custom feature for better handling. The insert 118 may be made from a material, such as, but not limited to polyoxymethylene (POM). A valve fixture, made from tinplate or aluminum, for example, and may have an outer diameter of at least 32 mm. The combination of the valve 102, insert 118, and actuator 116 achieves a fine mist spray with a consistent pattern and output.

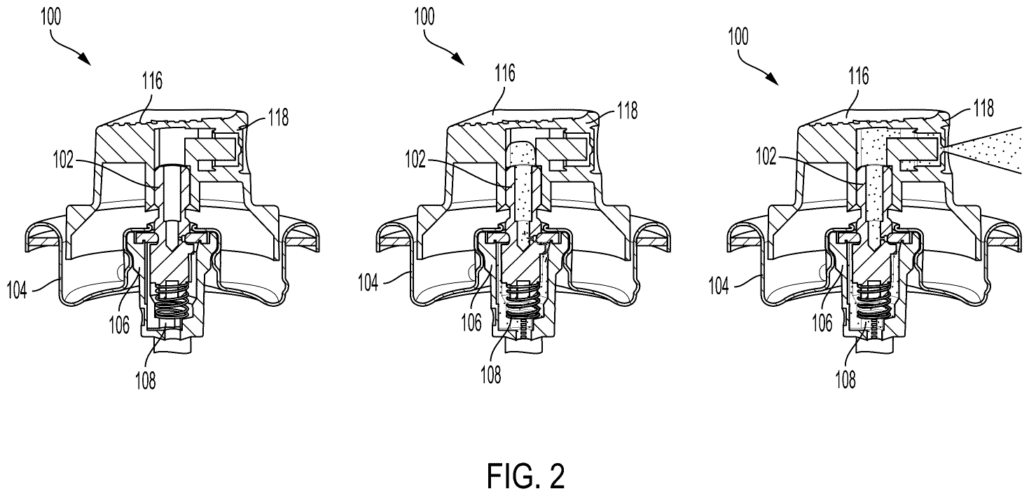

FIG. 2 illustrates how the spray composition moves through the spray assembly 100. In the left image, the spray assembly 100 is in a resting state. The liquid product is not yet entering the valve 102, and it is not being sprayed. The spring 108 and actuator 116 are in their default positions. In the middle image, the composition flows into the valve 102 and moves upwards. As the composition moves through the fluid path 117 of the actuator 116, the insert 118 helps to guide the composition, and to produce the desired spray characteristics. In the right image, the composition flows out of the aperture 120 of the insert. The composition is expelled as a sprayed mist, as the propellant evaporates almost immediately.

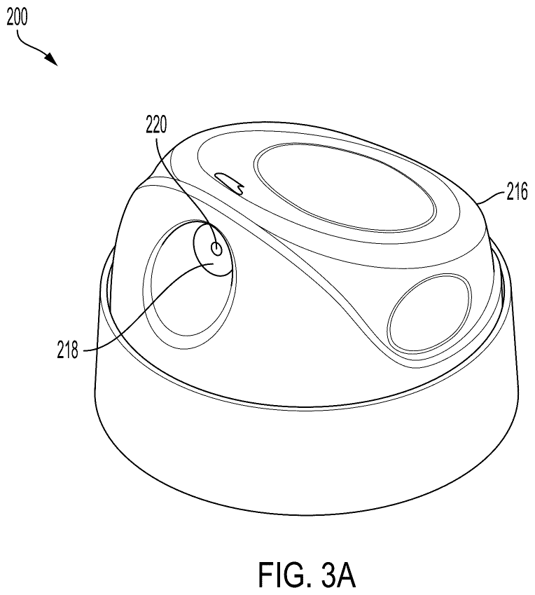

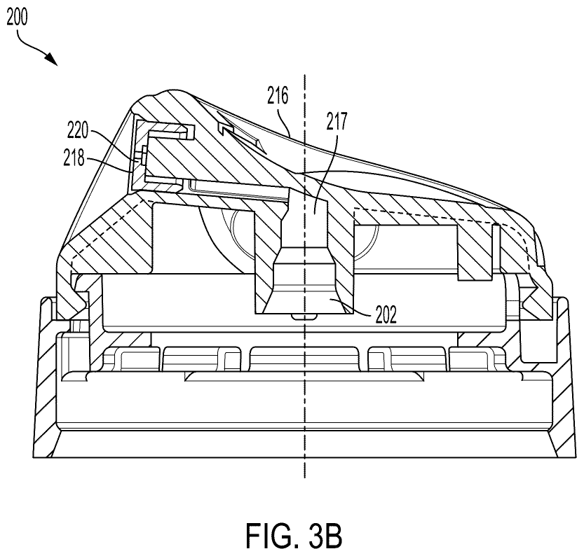

FIGS. 3A and 3B illustrate a spray assembly 200 including an actuator 216 and an insert 218 which has an aperture 220 for dispensing a spray formulation into a mist. FIG. 3B is a cross-sectional view of the spray assembly 200. The actuator 216 includes a fluid path 217 connecting an interior of a container to the aperture 220 when the actuator 216 is pressed. The spray assembly 200 may be used to operate when it is upside-down or upright. The ability of a spray assembly to operate upside-down is important for use in a postpartum pain spray for ease of operation.

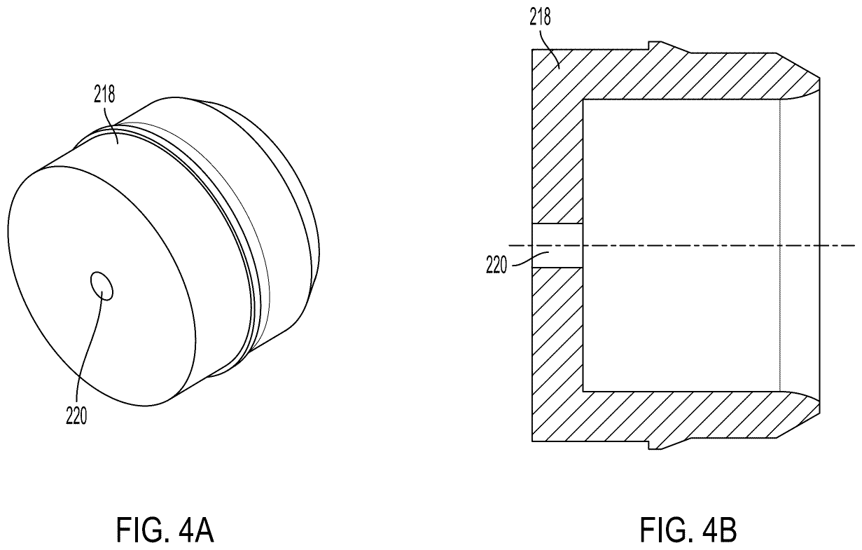

FIGS. 4A and 4B illustrate insert 218, as described above. The aperture 220 of the insert 218 is sized to provide the desired mist properties, such as a fine mist. The size of the aperture may be selected according to the particular spray formulation. The aperture size may have a diameter of about 0.005 to 0.05, including about 0.007 to about 0.025.

The dimensions and materials described for the sprayer assembly are exemplary dimensions and materials. Aspects of the present disclosure are not limited to these dimensions, as other dimensions are contemplated.

Given the spray formulation of the present disclosure may include up to 10% analgesic and up to 10% skin protectant, the amount of propellant provided cannot be a high percentage by weight of the spray formulation. Therefore, the present disclosure provides a spray composition that is capable of being sprayed as a mist using various spray assemblies.

### Brookfield Viscosity Test Method

The viscosity of samples can be measured using a standard viscometer, such as, for example, a Brookfield DV2T viscometer (manufactured by Brookfield Ametek, Middleboro, MA, US), fitted with a helipath disc shaped or cylindrical spindle. The viscometer is leveled, set up and calibrated according to the manufacturer's standards. The viscometer speed (RPM) is selected to ensure the measured viscosity is within the manufacturer's recommended settings (e.g., 5 RPM).

Samples are stored in sealed glass jars with an opening and internal diameter of at least 40 mm and filled to a height of at least 50 mm with care taken to avoid entrapped air bubbles. Centrifugation may be used to help removed entrained gas or air. Sample jars are equilibrated at 23° C.±2° C. and about 50%+2% relative humidity for at least 24 hours prior to measurement.

Viscosity is measured at 23° C.±2° C. and about 50%±2% relative humidity by placing the uncapped sample jar under the viscometer and lowering the viscometer until the top of the disc or cylinder touches the surface of the sample. The descending helipath is turned on and a timer started once the top of the disc or cylinder touches the surface of the sample. A reading is taken about every 10 seconds over the time period of between about 45 seconds and about 1 minute. The viscosity is calculated as the arithmetic average of the viscosities recorded. Care is taken to ensure the spindle does not touch the glass jar.

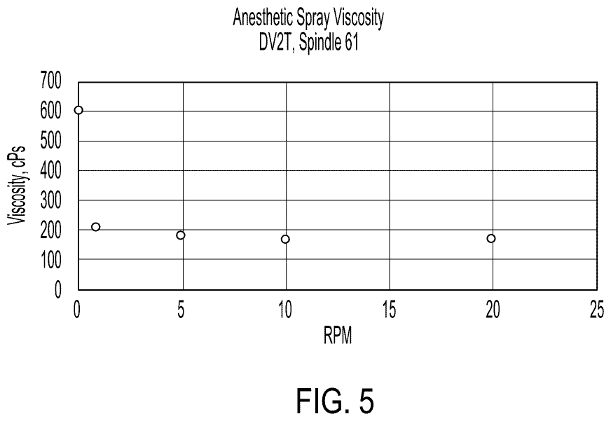

FIG. 5 is a viscosity chart showing the measured viscosity against the revolutions per minute (RPM) of the viscometer. Example 1, as shown in Table 1, has a higher viscosity at 0.1 RPM, and the viscosity measurements are lower as the RPM increases. In rheology, such fluid behavior is referred to as “shear thinning”, which is the non-Newtonian behavior of fluids whose viscosity decreases under shear strain. Examples of fluids that exhibit shear thinning include ketchup, which flows as the speed when squeezed from a container, but generally retains a shape once sprayed onto a surface. Such fluid behavior is due to the polymeric components and results in small droplets when the spray formulation is exiting the spray assembly. The high viscosity at low RPM indicates that the formulation will adhere to the targeted skin area to provide the desired cooling and skin protectant functions.

This high viscosity measurement at 0.1 RPM indicates that the formulation will have good static stability and resist separation. Also, FIG. 5 shows a flattening of the viscosity measurement as RPM increases, which indicates that the formulation will result in small droplets, with a narrow droplet size distribution and even coverage of the targeted spray area.

### Surface Tension Test Method

In accordance with EU A.5 Regulation (EC) No 440/2008, Annex, Official Journal of the European Union L142/1, surface tension was measured by the plate method (OECD Guidelines for The Testing of Chemicals, 115, Jul. 27 1995).

Prior to testing the sample, the surface tension of Barnstead GenPure Pro water was tested for verification purposes. The surface tension of water was measured at 72.58 mN/m at 22.4° C., which is within the acceptable tolerance of the literature value (72.37 mN/m@22.5° C.).

Surface tension was measured on a single sample in duplicate (each measurement is an average of 10 sequential analyses) by the Wilhelmy plate method on a Krüss K11 Tensiometer. In these experiments, a platinum rod probe is hung on a balance and brought into contact with the liquid interface. The forces experienced by the balance as the probe interacts with the surface of the liquid can be used to calculate surface tension. The probe used is a platinum rod, and has a wetted length of 6.283 mm. The instrument resolution is 0.01 mN/m. For the plate method a correction calculation is not necessary as per the instrument manual. The surface tension is calculated using the following equation:

$$σ = F / L \cdot cos θ$$

where σ=surface tension: F=force acting on the balance: L=wetted length: θ=contact angle.

The sample was found to not be soluble in water, so it was tested without any dilution. The sample was analyzed at 20° C. The temperature of the sample was obtained using a NIST traceable thermometer, with an accuracy of ±0.2° C. (Serial #230859189, retest date Dec. 19, 2025), immediately before the surface tension measurement. Each surface tension measurement consists of data collected over 10 seconds at 1 second intervals, with the average surface tension reported.

The surface tension of the formulation may be less than the surface tension of water (72 mN/m). In some formulations, the surface tension may be about less than 50 mN/m at about 20 to 25° C. Lower surface tension reduces the cohesive forces at the liquid's surface, allowing the liquid to break up more easily into smaller droplets during spraying. This results in finer droplets, providing better coverage and more efficient use of the sprayed composition. Lower surface tension can increase surface wetting, improving adhesion and effectiveness of the sprayed composition on skin.

The ingredients and the composition described are exemplary. Aspects of the present disclosure are not limited to specific compositions and/or ingredients, as other ingredients and compositions are contemplated. In some examples, an amount of the propellent may be greater than 50%. For example, the amount of propellent may be 65% (or greater). In other examples, the propellent may be less than 50%.

As discussed, various aspects of the present disclosure provide a dual-action anesthetic healing spray uniquely formulated and delivered for the relief of pain and promotion of healing in sensitive skin regions, particularly the perineal area following childbirth. Various aspects combine a local anesthetic, such as benzocaine, at clinically effective concentrations with one or more skin protectants, including glycerin, petrolatum, and mineral oil, in a sprayable composition. The composition further includes a polyol-based carrier and a propellant, preferably dimethyl ether, which together provide solubility, stability, and spray performance. The formulation is specifically engineered to deliver a fine, even mist while functioning reliably when the spray container is held upside-down, thereby permitting hygienic, hands-free use by postpartum patients. The spray exhibits shear-thinning rheological properties, ensuring static stability in the container while producing small, uniform droplets that adhere to the skin without excessive run-off. Various aspects further encompass specialized spray assemblies incorporating a valve, actuator, and insert designed to dispense the composition at consistent rates and droplet sizes, even when the container is inverted. Unlike conventional sprays, the present invention avoids the inclusion of alcohols, essential oils, and other potential irritants, providing a cooling, non-irritating, and fast-drying spray tailored for use on highly sensitive tissue. Together, these features provide a composition and delivery system that address the shortcomings of prior postpartum sprays and achieve reliable pain relief and enhanced healing in a safe, user-friendly format.

In some examples, the anesthetic component is not limited to benzocaine but may include other local analgesics such as lidocaine, pramoxine, dyclonine, capsaicin, menthol, or salicylates, either alone or in combination. In some examples, benzocaine may be combined with lidocaine to provide multi-modal pain relief with differing onset and duration profiles. In further examples, the composition may additionally include anti-inflammatory agents such as bisabolol, hydrocortisone acetate, or ginger root extract to reduce swelling, or antimicrobial agents such as benzalkonium chloride, chlorhexidine, or silver particles to provide infection prevention. In some embodiments, the carrier system may comprise solubilizing agents including cyclodextrins, liposomes, or nanoemulsions to enhance delivery of the anesthetic and protectants. The propellant is not limited to dimethyl ether; other acceptable propellants may include isobutane, propane, hydrofluoroalkanes, or compressed air and nitrogen. In some examples, the spray assembly may comprise a bag-on-valve system or barrier-separation packaging to ensure propellant and formulation stability and to permit 360° spraying. The spray actuator may also be configured to deliver metered doses or continuous spray, and the insert aperture may be varied to generate droplet size distributions tailored for different uses. In some example, the spray is applied to skin conditions beyond postpartum perineal pain, including hemorrhoids, anal fissures, insect bites, minor cuts, abrasions, burns, post-procedure discomfort, or general pain relief in sensitive areas.

In some examples, a process for manufacturing the anesthetic healing spray includes preparing a formulation by combining benzocaine with a skin protectant and a carrier to form a uniform solution or dispersion. The formulation is then introduced into an aerosol can along with a propellant, such as dimethyl ether, in a predetermined ratio to ensure sprayability and stability. Once the can is filled, a spray assembly is secured to the can by crimping, creating a sealed container capable of dispensing the composition as a mist. The spray assembly may include a valve, actuator, gasket, spring, insert, and dip tube configured to operate when the container is inverted. The result is a finished product that is shelf-stable, portable, and ready for use in postpartum pain relief and skin healing.

In some examples, a process of providing pain relief and skin protection includes supplying a user with a container pre-filled with the anesthetic healing composition comprising benzocaine, a skin protectant, a carrier, and a propellant. The container incorporates a spray assembly configured to atomize and dispense the composition as a fine mist. When the spray is applied to the desired area of skin, such as the perineum, the composition delivers a localized cooling and numbing sensation due to the benzocaine, while simultaneously depositing a skin protectant layer that promotes tissue healing and moisture retention. The spray assembly is specifically designed to function in an upside-down position, enabling hygienic, hands-free application to sensitive regions of the body.

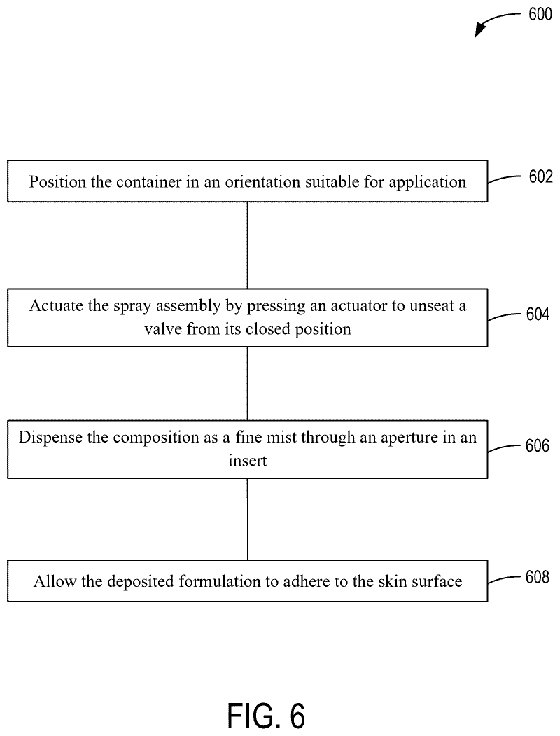

FIG. 6 is a flow diagram illustrating an example of a process 600 for providing pain relief and skin protection, in accordance with various aspects of the present disclosure. The process 600 may be used in conjunction with a sealed aerosol container having a composition that includes benzocaine in an amount of about 1 to 10%, a skin protectant in an amount of about 1 to 25%, a carrier, and a propellant. The container may further include a spray assembly configured to dispense the composition as a mist. The process 600 may be performed by a user, such as a human. As shown in the example of FIG. 6, the process 600 begins at block 602, wherein the user positions the container in an orientation suitable for application. The spray assembly is capable of dispensing even when the container is held upside-down. At block 604 the user actuates the spray assembly by pressing an actuator to unseat a valve from its closed position, thereby releasing the composition into a fluid path within the actuator. At block 606, the container dispenses the composition as a fine mist through an aperture in an insert. The propellant may rapidly evaporate to create a cooling sensation. The mist dispended from the container may be applied directly to the desired area of skin, which in certain embodiments includes the perineal region, to provide localized analgesia and skin protection. At block 608, the user allows the deposited formulation to adhere to the skin surface. The benzocaine provides temporary pain relief while the skin protectant promotes healing and maintains hydration

As used herein, the term “determining” encompasses a wide variety of actions. For example, “determining” may include calculating, computing, processing, deriving, investigating, looking up (e.g., looking up in a table, a database, or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A composition comprising:
1 to 10% benzocaine;
1 to 25% of a skin protectant selected from the group consisting of petrolatum, mineral oil, glycerin, or a combination thereof;
at least 1% propellant; and
a carrier comprising a surface tension modifier.

2. The composition of claim 1, wherein the benzocaine is present in an amount of 10% by weight of the composition.

3. The composition of claim 1, wherein the skin protectant is present in an amount of 10% by weight of the composition.

4. The composition of claim 1, wherein the skin protectant is glycerin and the glycerin is present in amount of about 1 to 10%.

5. The composition of claim 1, wherein the carrier is a polyol.

6. The composition of claim 1, further comprising one or more skin conditioning agents.

7. The composition of claim 6, wherein the one or more skin conditioning agents include one or more of aloe vera, witch hazel, or oat extract.

8. The composition of claim 1, wherein the propellant is dimethyl ether.

9. The composition of claim 8, wherein the propellant is about 50% of the composition by weight.

10. The composition of claim 9, wherein the carrier includes polyethylene glycol and PPG-3 methyl ether.

11. The composition of claim 1, wherein the composition does not include alcohol, lavender oil, and peppermint oil.

12. The composition of claim 1, wherein the composition is sprayed from a container having a sprayer assembly, the sprayer assembly comprising:
a valve coupled to the container via a valve fixture,
a housing coupled to the valve,
a gasket disposed between a portion of the valve and a section of the housing,
a spring configured to maintain the valve in a closed state,
an actuator configured to be pressed to unseat the valve from the closed state and to spray the composition, and
an insert coupled to the actuator, wherein the insert includes an aperture through which the composition passes through.

13. The composition of claim 12, wherein the spray assembly further comprises a dip tube coupled to the housing, wherein the dip tube extends downward from the housing, and the dip tube includes a first opening to draw the composition into the dip tube.

14. The composition of claim 13, wherein the spray assembly further comprises a ball that is configured to block a second opening when the container and spray assembly are positioned in a substantially upright position, and when the container and spray assembly are substantially upside-down position the ball moves away from the second opening to allow the composition to enter into the dip tube, wherein the second opening is proximal to the valve fixture of the spray assembly.

15. The composition of claim 14, wherein the spray assembly further comprises, wherein the valve has a height of at least 6 mm before crimping, and the insert has an aperture diameter of about 0.007 to 0.025 mm.

16. The composition of claim 11, wherein the spray assembly functions when the spray assembly is substantially upside-down.

17. A method for manufacturing the composition of claim 1, wherein the method includes:
forming a formulation including the benzocaine, the skin protectant, and the carrier; and
filling a can with the formulation and the propellant, and
sealing the can by crimping a spray assembly to the can.

18. A method of providing a pain relief and skin protectant to an area of skin, the method comprising:
providing a container having a composition including benzocaine, a skin protectant, a carrier, and a propellant, wherein the benzocaine is present in amount of about 1 to 10%, and the skin protectant is present in an amount of about 1 to 25%, and the container includes a spray assembly configured to spray the composition out of the container as a mist,
applying the mist to the area of skin to provide a cooling sensation and promote healing.

19. The method of claim 18, wherein the area of skin includes the perineum.

20. The method of claim 18, wherein the spray assembly includes
a valve coupled to a container via a valve fixture,
a housing coupled to the valve,
a gasket disposed between a portion of the valve and a section of the housing,
a spring configured to maintain the valve in a closed state,
an actuator configured to be pressed to unseat the valve from the closed state and to spray the composition,
and an insert coupled to the actuator, wherein the insert includes an aperture through which the composition passes through.
